(19) United States
(12) Patent Application Publication
(10) Pub. No.: US 2019/0124878 A1
(43) Pub. Date: May 2, 2019

(54) QUARTZ - WINTER OILSEED RAPE (Brassica Napus L emend)

(71) Applicant: KWS MOMONT RECHERCHE SARL, MONS EN PEVELE (FR)

(72) Inventor: THIERRY PIERRE ADRIEN MOMONT, Mons En Pevele (FR)

(21) Appl. No.: 15/798,421

(22) Filed: Oct. 31, 2017

(51) Int. Cl. A01H 6/20; A01H 1/04; A01H 5/04; A01H 5/10

(52) U.S. Cl. CPC A01H 6/20; A01H 5/10; A01H 5/04; A01H 1/04

(57) ABSTRACT

Quartz is a variety of winter oilseed rape that arose from a long-term breeding program focused on key output traits of (1) winter survival, (2) seed yield and (3) financial return for farmers in this order. Breeding objectives were restricted to these 3 so as these traits could be quickly identified.

Placement of the invention into research trials in environments that demanded strong expression of these traits quickly indicated the unique potential of this invention in a large geographic area of the US Central Plains. Commercialization of this invention in a logical manner throughout the region will bring previously unrealized benefits to US farmers and US oilseed crushing industry.

We, KWS Moment Recherche SARL, represented by Mr Thierry Momont, wishes to apply for a Utility Patent for its Winter Oilseed rape variety named QUARTZ.

Please find below its description

| Variety denomination | Quartz |
|---|---|
| Species | Winter Oilseed rape |
| Botanical name of taxon | Brassica napus L emend |
| Information on origin of the variety | Astrid x 99EA081 |
| Breeding method | Dihaploid |
| Place of discovery | North of France |
| Characters descriptions | States of Expression |
| Seed-erucic acid | Absent |
| Seed cotyledon-length | Medium |
| Seed cotyledon-width | Medium to broad |
| Seed cotyledon-lope separation | Medium to broad |
| Seed cotyledon-saddle depth | Medium to deep |
| Seed cotyledon-width/length | Medium to large |
| Seed cotyledon-lope separation/saddle depth | Small to medium |
| Leaf-green color | Medium to dark |
| Leaf-glaucosity | Present |
| Leaf-development of lobes | Present |
| Leaf-number of lobes | Medium |
| Leaf-dentation of margin | Medium to strong |
| Time of flowering | Medium |
| Flower-color of petals | Yellow |
| Flower-Length of petals | Medium |
| Flower-width of petals | Medium |
| Production of pollen | Present |
| Plant-height at full flowering | Medium |
| Plant-total length including side branches | Medium |
| Top shoot-length | Medium |
| Siliqua-length (between peduncle and beak) | Short to medium |
| Siliqua-width | Narrow to medium |
| Siliqua-length/width | Medium |
| Siliqua-length of beak | Short to medium |
| Siliqua-length of peduncle | Medium |

The rapeseed Quartz has been bread using the Double Haploid methods. This process involves obtaining haploid plants from reproductive cell-bearing organs, called male or female gametophytes, and returning to the diploid phase.

1- Quartz has a unique growth habit that confers adaptation to continental type climates such as the central US plains, especially due to its excellent winter hardiness.

2- Having delivered some remarkable on-farm performance over the years, Quartz remains a popular variety with farmers in regions of the world growing winter oilseed rape (Winter Canola)

3- Quartz, is a winter oilseed rape that offers very balanced agronomical traits. A vigorous growth habit from the start, his medium stiff straw makes it easy to harvest. Quartz offers outstanding resistance for both phoma and lodging. It also possess a good oil content.

4- Reliable performance in US University and Private Trials. Quartz has shown very good yield results in the trial network organized by Michael J. Stamm from the Kansas State University (see attached results)
